(19) United States
(12) Patent Application Publication
(10) Pub. No.: US 2022/0064704 A1
(43) Pub. Date: Mar. 3, 2022

(54) METHODS OF PREPARING LARGE QUANTITIES OF SINGLE-STRANDED DNA (ssDNA)

(71) Applicant: Applied Materials, Inc., Santa Clara, CA (US)

(72) Inventors: Chung Pui Cheng, Singapore (SG); Tze Howe Charn, Singapore (SG)

(21) Appl. No.: 17/410,925

(22) Filed: Aug. 24, 2021

Related U.S. Application Data
(60) Provisional application No. 63/072,064, filed on Aug. 28, 2020.

(51) Int. Cl. C12Q 1/6806; C12N 15/10; C12Q 1/6841

(52) U.S. Cl. CPC C12Q 1/6806; C12Q 1/6841; C12N 15/1013

(57) ABSTRACT

The disclosure relates to methods of preparing single-stranded DNA (ssDNA). The ssDNA can be used, for example, to prepare functionalized alignment beads as fiducial markers to improve image registration in fluorescence assays for the detection and quantitation of analytes in a sample.

### CROSS-REFERENCE TO RELATED APPLICATIONS

This application claims benefit under 35 U.S.C. § 119(e)(1) of U.S. Provisional Application No. 63/072,064, filed on Aug. 28, 2020, which is incorporated by reference herein.

### TECHNICAL FIELD

The disclosure relates methods of preparing single-stranded DNA (ssDNA). More specifically to methods to efficiently prepare large quantities of ssDNA at a low cost. The ssDNA can be used, for example, to prepare functionalized alignment beads as fiducial markers to improve image registration in fluorescence assays for the detection and quantitation of analytes in a sample.

### BACKGROUND

In amplification by polymerase chain reaction (PCR), two primers are designed to hybridize to the template DNA at positions complementary to respective primers that are separated on the DNA template molecule by some number of nucleotides. The base sequence of the template DNA between and including the primers is amplified by repetitive complementary strand extension reactions whereby the number of copies of the target DNA fragments is increased by several orders of magnitude. Amplification is exponential as 2n, where n equals the number of amplification cycles.

In vitro transcription and reverse transcription (IVT & RT) is a method that involves three steps: preparation of dsDNA templates, transcription of RNA from the dsDNA, and preparation of ssDNA from the RNA. Specifically, the dsDNA template is converted to an RNA via transcription, and then the RNA is reverted back to ssDNA using a reverse transcriptase. For in vitro transcription, PCR products or plasmids (containing a restriction site) can be used as the dsDNA templates, and transcription can be performed using a T7 promoter and the very strong T7 RNA polymerase. The RNA template can be cleaved using RNase H, which leaves a heteroduplex structure at the 3′ end of the DNA. IVT & RT can be used to synthesize ssDNA of various lengths.

Nucleic acid purification and clean-up are mandatory for genomic applications including sequencing, qPCR/ddPCR/PCR, microarrays and other enzymatic reactions, in order to separate the nucleic acid product from the enzymes, nucleotides, primers and buffer components. For example, purification of DNA from a PCR reaction is typically necessary for downstream use, and facilitates the removal of enzymes, nucleotides, primers and buffer components. Commonly used methods employ spin columns containing a silica matrix to which DNA can be selectively bound in the presence of chaotropic salts. Other reaction components, such as enzymes, nucleotides, detergents, and primers either do not bind well or are removed during the wash steps. Finally, the DNA is eluted from the columns under low-salt conditions and is ready for demanding downstream applications. In addition, the solid phase reversible immobilization (SPRI) technology can be used to maximize recovery, consistency and speed. For example, small nucleic acids can be enriched using a solid support such as a bead (e.g., a paramagnetic bead comprising carboxylate groups).

DNA is instrumental to myriad applications in biological imaging, bionanotechnology and synthetic biology. Many of these applications rely on the availability of ssDNA. For example, ssDNA is required in CRISPR/Cas9-mediated homology directed repair (HDR), fluorescent in-situ hybridization (FISH) imaging and DNA-origami folding. Depending on the required size, scale and purity, the production of ssDNA can become prohibitively expensive or onerous. Although chemically synthesized ssDNA is commercially available, such ssDNA is expensive, limited beyond lengths of ˜200 nt and requires additional processing to remove impurities. Alternatively, production of single strands from a double-stranded DNA (dsDNA) template via enzymatic processing, micro-bead sequestration, rolling circle amplification, asymmetric polymerase chain reaction (PCR) and co-polymerization and electrophoresis methods may be used, but is frequently limited by complexity of the protocols, scalability and/or purity of the recovered strands.

Many applications require large quantities of ssDNA. For example, to prepare functionalized alignment beads as fiducial markers to improve image registration in fluorescence assays, a large quantity of ssDNA (preferably several milligrams) is needed, especially when the goal is to image tissue sections. However, the through-put of existing methods to prepare ssDNA is limited by the through-put of DNA purification columns, which is generally limited to 100 per column. This is not enough to satisfy the need for preparing functionalized alignment beads as fiducial markers to improve image registration in fluorescence assays, especially when the goal is to image tissue sections. In addition, the dead volume in commercial DNA purification spin columns leads to inefficiency. Thus, there is a need for a preparation method that can generate a large quantity of ssDNA in a more efficient manner.

### SUMMARY

In one aspect, a method of generating single-stranded DNA (ssDNA) includes providing a plurality of DNA oligonucleotides, performing a polymerase chain reaction (PCR) amplification of the DNA oligonucleotides, purifying the PCR products with magnetic beads, performing in vitro transcription (IVT) reaction with the PCR products to generate intermediate RNA molecules, performing reverse transcription (RT) of the intermediate RNA molecules to generate ssDNA, and purifying the ssDNA with magnetic beads.

This application describes a novel method for preparing large quantities of single-stranded DNA (ssDNA) in an efficient manner. In one aspect, the current method uses a polymerase chain reaction (PCR) to amplify DNA oligonucleotides, purifies the PCR product using magnetic beads containing carboxylate groups, and then subjects the purified PCR product to in vitro transcription (IVT) to generate amplified RNA (IVT product). ssDNA oligonucleotides are then transcribed from the IVT product via reverse transcription (RT) and purified using magnetic beads comprising carboxylate groups. This workflow is capable of generating large quantities of ssDNA of various lengths (e.g., 35-1000 bp).

The subject matter described may result in, but is not limited to, one or more of the following advantages.

The present method uses polymerase chain reaction (PCR) with a single PCR protocol to amplify a mixture of DNA oligonucleotides with different sequences. Traditionally, PCR reaction requires designing different PCR protocols for different primer pairs because different primer sequences have different melting temperature. By using a single PCR protocol to amplify a mixture of DNA oligonucleotides with different sequences, the current methods greatly enhance DNA amplification efficiency. In addition, the incorporation of the PCR amplification reduces overall costs to generate the same amount of ssDNA because PCR reagents are cheaper than IVT reagents. Comparing to DNA purification spin columns, magnetic beads comprising carboxylate groups greatly enhance the through-put of DNA purification, in part because the binding area is larger and there is no dead volume. For example, starting from 1-10 ng DNA oligonucleotides the current method can yield about 1000-10,000 μg purified ssDNA in one run. In addition, magnetic beads comprising carboxylate groups offer the additional advantage of size selection. Another advantage of the present method is that it is relatively easy to automate purification based on magnetic beads because there is no need for centrifugation.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### BRIEF DESCRIPTION OF THE DRAWINGS

FIG. 1 is an illustrative flow chart of the method of preparing ssDNA.

FIGS. 2A-2C depict an exemplary DNA ScreenTape analysis on the PCR product, RVT product and RT product starting from 6.4 ng DNA oligonucleotides.

FIG. 3 depicts an alignment bead functionalized with nine different binding sites and labeled with three probes of different color dyes.

FIG. 4 depicts a multiplexed fluorescent in-situ hybridization (mFISH) imaging and image processing apparatus.

FIG. 5 is a flow diagram of a process for registering a first image and a second image.

FIG. 6 illustrates a flow chart of a process of data processing in which the processing is performed after images have been acquired.

### DETAILED DESCRIPTION

This present application describes a novel method for preparing single-stranded DNA (ssDNA) in an efficient manner. Existing methods of preparing ssDNA are hampered by low throughput of DNA purification, which is generally limited to 100 μg per column. Running multiple columns in parallel still provides only minimal quantities of ssDNA, and would be prohibitively expensive. In addition, the efficiency of commercial DNA purification spin columns is limited by the dead volume of the columns. Magnetic beads with carboxylate groups enable efficient, high through-put DNA purification, in part because the binding area is larger and there is no dead volume.

The present method uses polymerase chain reaction (PCR) with a single protocol to amplify a mixture of DNA oligonucleotides with different sequences. Traditionally, PCR reaction requires designing different PCR protocols for different primer pairs because different primer sequences have different melting temperature. By using a single PCR protocol to amplify a mixture of DNA oligonucleotides with different sequences, the current methods greatly enhance DNA amplification efficiency. Coupling this amplification with IVT & RT and magnetic bead purification greatly increases the yield of ssDNA per reaction. For example, starting from 1-10 ng DNA oligonucleotides the current method can yield about 1,000-10,000 μg purified ssDNA. In addition, magnetic beads with carboxylate groups offer the additional advantage of size selection. Another advantage of the present method is that it is relatively easy to automate purification based on magnetic beads because there is no need for centrifugation. Finally, the present method reduces costs by incorporating the PCR amplification because PCR reagents are cheaper than IVT reagents.

The ssDNA described in the present application are ready for immediate use in various applications. For example, to prepare functionalized alignment beads as fiducial markers to improve image registration in fluorescence assays. This requires a large quantity of ssDNA (preferably several milligrams), especially when the goal is to image tissue sections—this quantity of ssDNA would be time consuming and expensive to generate using traditional methods.

In one aspect, the present invention provides a method of generating ssDNA, the method comprising:

(a) Providing a plurality of DNA oligonucleotides;

(b) Performing a polymerase chain reaction (PCR) amplification of the DNA oligonucleotides;

(c) Purifying the PCR products with magnetic beads;

(d) Performing in vitro transcription (IVT) reaction with the PCR products to generate intermediate RNA molecules;

(e) Performing reverse transcription (RT) of the intermediate RNA molecules to generate ssDNA;

(f) Purifying the ssDNA with magnetic beads.

In some embodiments, the PCR amplification in step (b) uses a single annealing temperature to amplify DNA oligonucleotides with different sequences in one working solution.

In some embodiments, the magnetic beads in steps (c) and (f) comprise carboxylate groups.

In some embodiments, the magnetic beads in steps (c) and (f) provide DNA size selection.

In some embodiments, the starting DNA oligonucleotides in step (a) is about 1-10 ng and the purification in step (d) yields about 1,000-10,000 μg IVT product.

In some embodiments, the starting DNA oligonucleotides in step (a) is about 1-10 ng and the purification in step (f) yields about 700-7,000 μg purified ssDNA.

As used herein, the term “about” is used to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

The term “each,” when used in reference to a collection of items, is intended to identify an individual item in the collection but does not necessarily refer to every item in the collection, unless expressly stated otherwise, or unless the context of the usage clearly indicates otherwise.

In some embodiments, DNA oligonucleotides with sequences for use in preparing functionalized alignment beads as fiducial markers to improve image registration in fluorescence assays are designed via a computational design step and generated by methods known in the field. In some embodiments, DNA oligonucleotides are generated via chemical synthesis. In some embodiments, DNA oligonucleotides are synthesized via enzymatic synthesis. In some embodiments, these DNA oligonucleotides are designed based on the sequence of the RNA target that they are designed to bind. In some embodiments, these DNA oligonucleotides are designed based on the desired binding affinity between the alignment bead and the corresponding reporting probe.

In some embodiments, the PCR amplification in step (b) uses multiple primer pairs with different sequences. In some embodiments, multiple DNA oligonucleotides with different sequences are amplified in the same working solution for PCR amplification in step (b).

In some embodiments, the PCR amplification in step (b) uses a single annealing temperature for multiple primer pairs with different sequences. In some embodiments, the PCR amplification in step (b) uses an annealing temperature that ranges from about 60° C. to about 70° C. In some embodiments, the PCR amplification in step (b) uses an annealing temperature of 66° C. In some embodiments, the PCR in step (b) is a limited-cycle PCR.

In some embodiments, the purification steps (c) and (f) use the same magnetic beads.

In some embodiments, the purification step (c) purifies about 1 μg to about 10 μs of DNA oligonucleotides.

In some embodiments, the magnetic beads in purification step (c) can provide DNA size selection. In some embodiments, the magnetic beads in purification step (c) selects DNA molecules ranging from about 50 bp to about 150 bp.

In some embodiments, the purification step (f) purifies about 100 μg to about 10 mg of ssDNA.

In some embodiments, the magnetic beads in purification step (f) can provide DNA size selection. In some embodiments, the magnetic beads in purification step (f) selects DNA molecules ranging from about 50 bp to about 150 bp.

In some embodiments, the RNA product is purified using magnetic beads after step (d) and before (e).

In some embodiments, the intermediate RNA molecules are removed after step (e) and before step (f). In some embodiments, the intermediate RNA molecules are removed in a pre-purification step. In some embodiments, the intermediate RNA molecules are removed through hydrolysis. In some embodiments, the remaining intermediate RNA molecules are removed by alkaline hydrolysis after step (e) and before step (f). In some embodiments, the remaining intermediate RNA molecules are removed by alkaline hydrolysis at a temperature ranging from about 60° C. to about 70° C. after step (e) and before step (f). In some embodiments, the remaining intermediate RNA molecules are removed by alkaline hydrolysis at a temperature of 65° C. after step (e) and before step (f). In some embodiments, the remaining intermediate RNA molecules are removed by RNase treatment. In some embodiments, the remaining intermediate RNA molecules are removed by incubating the RT product with RNase A (100 mg/ml) for 2 minutes at room temperature.

In some embodiments, the ssDNA range from about 35 bp to about 1000 bp in length, for example, about 35 bp to about 150 bp, about 75 bp to about 250 bp, about 150 bp to about 300 bp, about 250 bp to about 500 bp, about 350 to about 600 bp, about 500 to about 750 bp, about 650 to about 900 bp, or about 750 to about 1000 bp.

In some embodiments, the magnetic beads in step (c) contain carboxylate groups. In some embodiments, the magnetic beads in step (c) are AMPure XP beads. In some embodiments, the magnetic beads in step (f) contain carboxylate groups. In some embodiments, the magnetic beads in step (f) are AMPure XP beads.

In some embodiments, the reverse transcription in step (e) is performed at a temperature ranging from about 60° C. to about 70° C. In some embodiments, the reverse transcription in step (e) is performed at a temperature of 65° C.

In some embodiments, the starting DNA oligonucleotides in step (a) is about 0.1 ng to about 10 ng, about 0.1 ng to about 2 ng, about 0.5 ng to about 3 ng, about 1 ng to about 4 ng, about 2 ng to about 5 ng, about 3 ng to about 6 ng, about 4 ng to about 7 ng, about 5 ng to about 8 ng, about 6 ng to about 9 ng, or about 7 ng to about 10 ng.

In some embodiments, the purified PCR product in step (c) is about 0.1 μg to about 10 μg, about 0.1 μg to about 2 μg, about 0.5 μg to about 3 μg, about 1 μg to about 4 μg, about 2 μg to about 5 μg, about 3 μg to about 6 μg, about 4 μg to about 7 μg, about 5 μg to about 8 μg, about 6 μg to about 9 μg, or about 7 μg to about 10 μg.

In some embodiments, the purified RVT product in step (d) is about 0.1 mg to about 10 mg, about 0.1 mg to about 2 mg, about 0.5 mg to about 3 mg, about 1 mg to about 4 mg, about 2 mg to about 5 mg, about 3 mg to about 6 mg, about 4 mg to about 7 mg, about 5 mg to about 8 mg, about 6 mg to about 9 mg, or about 7 mg to about 10 mg.

In some embodiments, the purified ssDNA in step (f) is about 0.07 mg to about 7 mg, about 0.0.7 mg to about 1.4 mg, about 0.35 mg to about 2.1 mg, about 0.7 mg to about 2.8 mg, about 1.4 mg to about 3.5 mg, about 2.1 mg to about 4.2 mg, about 2.8 mg to about 4.9 mg, about 3.5 mg to about 5.6 mg, about 4.2 mg to about 6.3 mg, or about 4.9 mg to about 7 mg.

In some embodiments, the starting DNA oligonucleotides in step (a) is about 0.1-100 ng and step (d) yields about 100-100,000 μg IVT product. In some embodiments, the starting DNA oligonucleotides in step (a) is about 0.1-100 ng and the purification in step (f) yields about 70-70,000 μg purified ssDNA. In some embodiments, the starting DNA oligonucleotides in step (a) is about 0.1-10 ng and the purification in step (d) yields about 100-10,000 μg IVT product. In some embodiments, the starting DNA oligonucleotides in step (a) is about 0.1-10 ng and the purification in step (f) yields about 70-7,000 μg purified ssDNA. In some embodiments, the starting DNA oligonucleotides in step (a) is about 1-10 ng and the purification in step (d) yields about 1,000-10,000 μg IVT product. In some embodiments, the starting DNA oligonucleotides in step (a) is about 1-10 ng and the purification in step (f) yields about 700-7,000 μg purified ssDNA.

In some embodiments, the length of the ssDNA is about 5-5000 bp, for example. In some embodiments, the length of the ssDNA is about 10 bp to about 2000 bp, about 25 bp to about 2000 bp, about 35 bp to about 2000 bp, about 35 bp to about 1000 bp, about 35 bp to about 500 bp, about 35 bp to about 150 bp, or about 50 bp to about 150 bp.

In some embodiments, the starting DNA oligonucleotides in step (a) is about 6.4 ng and step (b) yields about 6060 ng PCR product. In some embodiments, the starting DNA oligonucleotide in step (a) is about 6.4 ng and step (d) yields about 5046 μg IVT product. In some embodiments, the starting DNA oligonucleotides in step (a) is about 6.4 ng and step (f) yields about 3570 μg ssDNA.

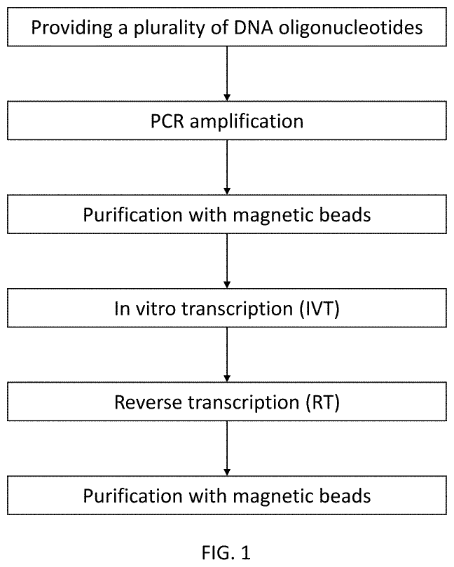

FIG. 1 is an illustrative flow chart of the method of preparing ssDNA. As shown in FIG. 1, the current method uses a polymerase chain reaction (PCR) to amplify a plurality of DNA oligonucleotides, purifies PCR using magnetic beads containing carboxylate groups, and then subjects the purified PCR product to in vitro transcription (IVT) to generate amplified RNA (IVT product). ssDNA is then transcribed from the IVT product via reverse transcription (RT) and purified using magnetic beads comprising carboxylate groups. This workflow is capable of generating large quantities of ssDNA of various lengths (e.g., 35-1000 bp) in an efficient manner.

Methods of Preparing Large Quantities of ssDNA

In some embodiments, DNA oligonucleotides with sequences for use in preparing functionalized alignment beads as fiducial markers to improve image registration in fluorescence assays are designed via a computational design step and generated by methods known in the field. In some embodiments, DNA oligonucleotides are generated via chemical synthesis. In some embodiments, DNA oligonucleotides are synthesized via enzymatic synthesis. In some embodiments, these DNA oligonucleotides are designed based on the sequence of the RNA target that they are designed to bind. In some embodiments, these DNA oligonucleotides are designed based on the desired binding affinity between the alignment bead and the corresponding reporting probe.

### PCR Amplification

In some embodiments, the DNA oligonucleotides are amplified by using a polymerase chain reaction (PCR). In some embodiments, DNA oligonucleotides with different sequences are mixed together for PCR amplification. In some embodiments, a single PCR protocol (and hence a single annealing temperature) is used for the mixture of DNA oligonucleotides with different sequences. In some embodiments, the PCR amplification uses an annealing temperature that ranges from about 60° C. to about 70° C. In some embodiments, the PCR amplification uses an annealing temperature of 66° C. In some embodiments, the DNA oligonucleotides are diluted to a working concentration of 0.4 ng/μL. In some embodiments, forward and reverse PCR primers are designed and generated using methods known in the art. In some embodiments, the forward and reverse PCR primers are diluted to 100 μM. In some embodiments, the PCR working solution is prepared according to Table 1. In some embodiments, the total volume of the PCR working solution is brought to 500 uL with Nuclease-Free Water. In some embodiments, volumes needed for each reagent for a total of 8 PCR reactions are mixed together. In some embodiments, 50 uL of the PCR working solution is added to each well in sterile (autoclaved) 8-strip PCR tubes 0.2 mL (e.g., Axygen, #14-222-250, or any equivalent) (“8-tube strips”). In some embodiments, the 8-tube strips are vortexed gently (low setting) in a vortex mixer (e.g., Corning, cat no. #10-320-807, or any equivalent) and centrifuged in a centrifuge (Eppendorf, cat no. #022621431, or any equivalent) to ensure the mastermix is not stuck on the cover of the 8-tube strips and that there are no bubbles. In some embodiments, the 8-tube strips are placed into the PCR thermocycler machine (ThermoFisher Scientific, SimpliAmp Thermal Cycler, or any equivalent) which is adjusted to carry out the PCR program in Table 2, which is designed for amplification of L1 oligo library.

In some embodiments, 1 uL of PCR product is diluted in 10× with Nuclease Free Water and saved to run DNA ScreenTape analysis using methods known in the art (e.g., using Agilent D1000 ScreenTape Assay and Reagents Agilent, #5067-5582, #5067-5583, #5067-5586, following manufacturer instructions). In some embodiments, a single, clean band confirms that there was specific product amplification. In some embodiments, the size of the PCR product is also verified by the ScreenTape analysis. In some embodiments, the concentration of the PCR product is also verified by the ScreenTape analysis based on peak graphs and the concentration should be around 10-50 ng/μl. In some embodiments, the PCR product is kept at 4° C. if it is not used immediately. In some embodiments, the samples are labeled with initials, library and date of amplification.

### PCR Product Ampure XP Beads Cleanup

In some embodiments, the PCR product is purified by magnetic beads containing carboxylate groups. In some embodiments, the PCR product is purified by Ampure XP beads (Beckman, #A63882) following manufacturer instructions. In some embodiments, 90 μl of Ampure XP beads is added into each well of 8-tube strips, mixed well, and incubated at room temperature for 5 minutes. In some embodiments, the 8-tube strips are placed on a magnetic stand (e.g., DynaMag™-2 Magnet Magnetic Stand, ThermoFisher Scientific, #12321D) until the liquid is clear (e.g., after 5 minutes). In some embodiments, this ensures that the Ampure XP beads are pulled down by magnetic force. In some embodiments, the 8-tube strips are removed from the magnetic stand and 140 μl supernatant from each well is discarded. In some embodiments, the Ampure XP beads are washed twice using the following protocol: a) adding 180 μl fresh 80% EtOH (ethanol) (Merck, #200-578-6) to each well; b) incubating on the magnetic stand for 30 seconds; c) removing and discarding all supernatant from each well. In some embodiments, the Ampure XP beads are then air-dried on the magnetic stand for 15 minutes. In some embodiments, the 8-tube strips are then removed from the magnetic stand. In some embodiments, 11 μl Nuclease Free Water is added to each well of the 8-tube strips, mixed thoroughly, and then incubated at room temperature for 2 minutes. In some embodiments, the 8-tube strips are placed on a magnetic stand until the liquid is clear (e.g., after 5 minutes). In some embodiments, 11 μl supernatant from each well is transferred into a separate 1.5 ml Eppendorf tube (e.g., Axygen, #MCT-175-C, or any equivalent). In some embodiments, 1 uL of the purified PCR product is diluted in 10× with Nuclease Free Water and saved to run DNA ScreenTape analysis using methods known in the art (e.g., using Agilent D1000 ScreenTape Assay and Reagents Agilent, #5067-5582, #5067-5583, #5067-5586, following manufacturer instructions).

### In Vitro Transcription (IVT)

In some embodiments, the PCR product is transcribed into RNA molecules via in vitro transcription. In some embodiments, the working solution for IVT is assembled following the recipe in Table 3. In some embodiments, 30 μl of the IVT working solution is added into each well of 8-tube strips. In some embodiments, the 8-tube strips are then placed into a PCR thermocycler machine and incubated at 37° C. overnight. In some embodiments, the IVT reaction is stopped by DNase treatment by adding 2 uL DNase (2 U/uL) and 20 uL Nuclease Free Water into each well of the 8-tube strips and incubating for 15 minutes. In some embodiments, each well of the 8-tube strips contains 30 uL of the IVT product, 20 uL Nuclease Free Water and 2 uL DNase (2 U/uL). In some embodiments, after the DNase treatment, 0.5 μl of 0.5M EDTA (final concentration 5 mM) is added to each well of the 8-tube strips and incubated at 75° C. for 10 minutes.

### IVT Product RNA Beads Cleanup

In some embodiments, the IVT product is purified using magnetic beads containing carboxylate groups. In some embodiments, the IVT product is purified by RNA beads (e.g., RNAClean XP from Beckman, Item No: A66514) following manufacturer instructions. In some embodiments, 94.5 μl of RNA beads is added into each well of the 8-tube strips, mixed well, and incubated at room temperature for 5 minutes. In some embodiments, the 8-tube strips are placed on a magnetic stand until the liquid is clear (e.g., after 5 minutes). In some embodiments, this ensures that the RNA beads are pulled down by magnetic force. In some embodiments, the 8-tube strips are removed from the magnetic stand and 140 μl supernatant from each well is discarded. In some embodiments, the RNA beads are washed twice using the following protocol: a) adding 180 μl fresh 80% EtOH (ethanol) to each well; b) incubating on the magnetic stand for 30 seconds; c) removing and discarding all supernatant from each well. In some embodiments, the RNA beads are then air-dried on the magnetic stand for 15 minutes. In some embodiments, the 8-tube strips are then removed from the magnetic stand. In some embodiments, 11 μl Nuclease Free Water is added to each well of the 8-tube strips, mixed thoroughly, and then incubated at room temperature for 2 minutes. In some embodiments, the 8-tube strips are placed on a magnetic stand until the liquid is clear (e.g., after 5 minutes). In some embodiments, 11 μl supernatant from each well is transferred into a separate 1.5 ml Eppendorf tube (e.g., Axygen, #MCT-175-C, or any equivalent). In some embodiments, 1 uL of the purified IVT product is diluted in 10× with Nuclease Free Water and saved to run RNA ScreenTape Analysis using methods known in the art (e.g., using Agilent TapeStation RNA ScreenTape & Reagents, #5067-5577, #5067-5578, #5067-5576, following manufacturer instructions). In some embodiments, 1 uL of the purified IVT product is diluted in 10× with Nuclease Free Water and saved to run on a PAGE gel following methods known in the art.

### Reverse Transcription (RT)

In some embodiments, ssDNA is generated from the purified IVT product via reverse transcription. In some embodiments, the PCR thermocycler is pre-heated to 65° C. and kept ready for subsequent step. In some embodiments, the working solution for RT is assembled following the recipe in Table 4. In some embodiments, after adding 36 μL Nuclease Free Water, 20 μL 25 mM dNTPs, 150 μL 100 uM Forward Primer and 80 μL IVT product, the working solution is heated at 65° C. for 5 min, and then put in ice immediately before adding the other components (5×RT buffer, RNasin Plus and Maxima H-Reverse Transcriptase). In some embodiments, 50 μl of the RT working solution is added into each well of 8-tube strips and then incubated in the PCR thermocycling machine at 50° C. for 1 hour. In some embodiments, the RT reaction is stopped by alkaline hydrolysis by adding 10 μL 0.5 M EDTA and 10 μL 1 M NaOH into each well of the 8-tube strips. In some embodiments, the 8-tube strips are incubated at 65° C. for 15 min and cooled on ice for 5 minutes. In some embodiments, 9.8 μL of 2 M HCl is added to each well of the 8-tube strips to step the alkaline hydrolysis. In some embodiments, after adding HCl, the pH of the solution is around pH 7-9.

### RT Product Ampure XP Beads Cleanup

In some embodiments, the RT product is purified using magnetic beads containing carboxylate groups. In some embodiments, the RT product is purified by Ampure XP beads (Beckman, #A63882) following manufacturer instructions. In some embodiments, 145 μl of Ampure XP beads (Beckman, #A63882) is added into each well of the 8-tube strips, mixed well, and incubated at room temperature for 5 minutes. In some embodiments, the 8-tube strips are placed on a magnetic stand (e.g., DynaMag™-2 Magnet Magnetic Stand, ThermoFisher Scientific, #12321D) until the liquid is clear (e.g., after 5 minutes). In some embodiments, this ensures that the Ampure XP beads are pulled down by magnetic force. In some embodiments, the 8-tube strips are removed from the magnetic stand and 140 μl supernatant from each well is discarded. In some embodiments, the Ampure XP beads are washed twice using the following protocol: a) adding 180 μl fresh 80% EtOH (ethanol) (Merck, #200-578-6) to each well; b) incubating on the magnetic stand for 30 seconds; c) removing and discarding all supernatant from each well. In some embodiments, the Ampure XP beads are then air-dried on the magnetic stand for 15 minutes. The beads should not be over-dried. In some embodiments, the 8-tube strips are then removed from the magnetic stand. In some embodiments, 11 μl Nuclease Free Water is added to each well of the 8-tube strips, mixed thoroughly, and then incubated at room temperature for 2 minutes. In some embodiments, the 8-tube strips are placed on a magnetic stand until the liquid is clear (e.g., after 5 minutes). In some embodiments, 11 μl supernatant from each well is transferred into a separate 1.5 ml Eppendorf tube (e.g., Axygen, #MCT-175-C, or any equivalent). In some embodiments, 1 uL of the purified RT product is diluted in 10× with Nuclease Free Water and saved to run DNA ScreenTape analysis using methods known in the art (e.g., using Agilent D1000 ScreenTape Assay and Reagents Agilent, #5067-5582, #5067-5583, #5067-5586, following manufacturer instructions). In some embodiments, the size and concentration of the purified RT product are checked by DNA ScreenTape analysis. In some embodiments, the concentration of the purified RT product is around 2000-5000 ng/μl. In some embodiments, the purified RT product is stored at −20° C.

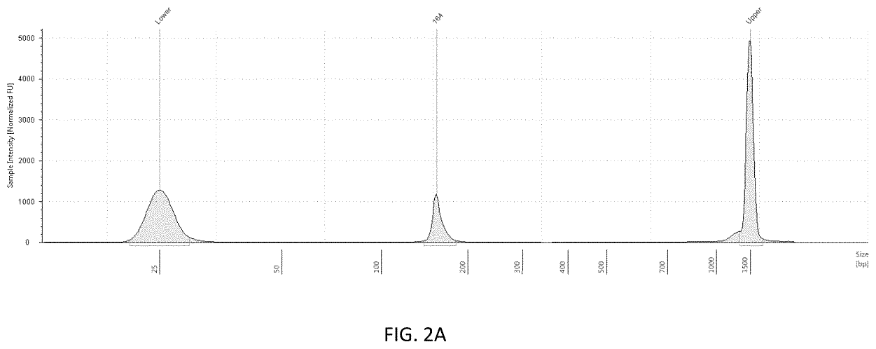

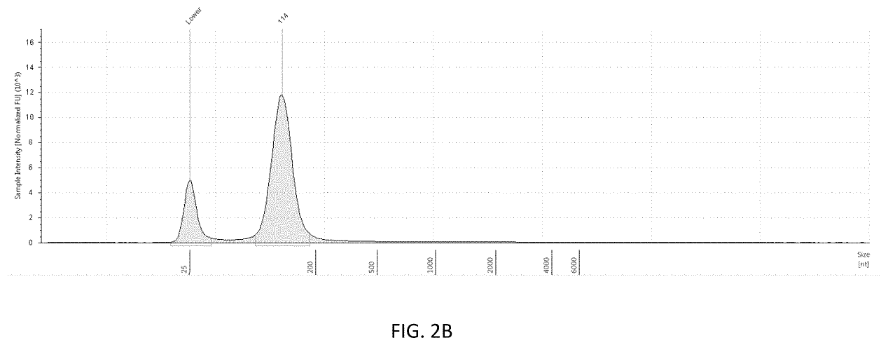

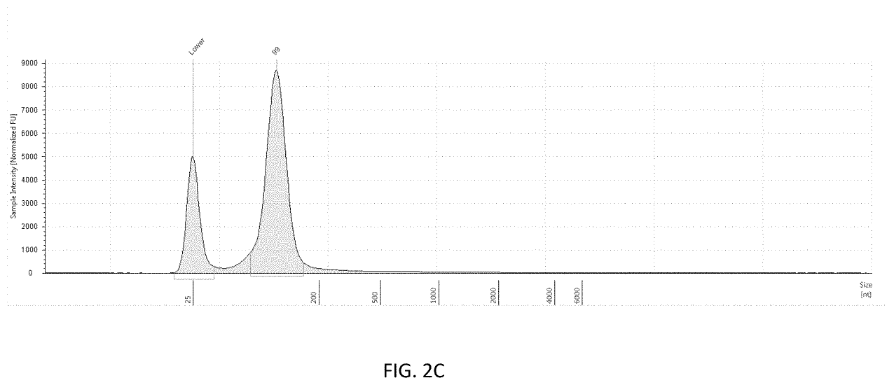

FIGS. 2A-2C depicts an exemplary DNA ScreenTape analysis on the PRC product, RVT product and RT product starting from 6.4 ng DNA oligonucleotides. FIG. 2A depicts the result of DNA ScreenTape analysis on the PCR product starting from 6.4 ng DNA oligonucleotides, showing 6060.0 ng PCR product (3030.0 ng/8-tube strips). FIG. 2B depicts the result of DNA ScreenTape analysis on the IVT product starting from 6.4 ng DNA oligonucleotides, showing 5046.0 μg IVT product (2523.0 μg/8-tube strips). FIG. 2C depicts the result of DNA ScreenTape analysis on the RT product starting from 6.4 ng DNA oligonucleotides, showing 3570.0 μg final ssDNA product (1785.0 μg/8-tube strips).

Some embodiments provide a method for performing an in situ fluorescence hybridization assay on a sample, the method comprising:

a) Contacting the sample with one or more targeting-probes, wherein each targeting-probe binds to an analyte in the sample, if present;

b) Contacting the sample with a plurality of fiducial markers, wherein each fiducial marker comprises a plurality of binding sites;

c) Contacting the sample with one or more readout-probes, wherein each readout-probe independently comprises a fluorescent moiety, and wherein each readout probe binds with the one or more targeting probes, if present, and the plurality of binding sites, thereby exhibiting one or more fluorescent signals;

d) Imaging the one or more fluorescence signals produced by each readout-probe;

e) Registering the image in step d);

f) Repeating steps (a)-(e) from 0 to 10 times;

g) Photobleaching the sample; and

h) Independently repeating steps (a)-(g) from 0 to 10 times.

In some embodiments, the fiducial markers comprise oligonucleotide-functionalized alignment beads. In some embodiments, single-color probes are used to label both in a single round of hybridization experiment, where the probes are designed to bind to both the analytes (such as nucleic acid molecules) and to the binding sites of the alignment beads (such as oligonucleotide binding sites). In some embodiments, multiple-color probes designed to hybridize to both the analytes and the binding sites of the alignment beads, as described herein, are used to label both in a single round of hybridization experiment. In some embodiments, a fiducial identification system can be used to identify alignment beads in a fluorescence microscopy image of a microscope slide comprising alignment beads and a sample, each having a common fluorescence wavelength. The fluorescence microscopy image depicts both the fiducial markers and the sample. In some embodiments, registering the image comprises a fiducial identification system capable of registering fluorescence images captured at different time points.

In some embodiments, each of the plurality of binding sites on the fiducial markers comprises an oligonucleotide. In some embodiments, each oligonucleotide is independently DNA or RNA. In some embodiments, each oligonucleotide is independently DNA. In some embodiments, each oligonucleotide is independently RNA. In some embodiments, each oligonucleotide is a ssDNA. In some embodiments, each oligonucleotide independently comprises 35-1000 residues.

In some embodiments, the plurality of fiducial markers comprise beads, wherein the beads comprise a bead core and a bead surface, wherein the surface comprises a plurality of binding sites. In some embodiments, the bead core comprises non-porous silica or an organic polymer. In some embodiments, the bead core comprises an organic polymer selected from polystyrene or polyisoprene.

In some embodiments, the alignment beads are not auto-fluorescent. In some embodiments, the alignment beads are not auto-fluorescent in about the same wavelength as any readout domain. In some embodiments, alignment beads comprise non-porous silica. In some embodiments, the alignment beads comprise one or more organic polymers. Examples of such organic polymers include, but are not limited to, polystyrene, polyethylene, polypropylene, and poly(vinyl)alcohol. In some embodiments, the alignment comprise a dispersed colloidal suspension of spherical particles comprising amorphous polyisoprene (latex).

In some embodiments, each targeting domain comprises an oligonucleotide. In some embodiments, each binding site comprises an oligonucleotide. In some embodiments, each analyte comprises an oligonucleotide. In some embodiments, the oligonucleotide is DNA. In some embodiments, the oligonucleotide is RNA. In some embodiments, the oligonucleotide is ssDNA. In some embodiments, each oligonucleotide independently comprises 35-1000 residues.

Attachment of the oligonucleotide binding sites to the alignment beads can be achieved via appropriate chemical coupling reactions which are well known to those skilled in the art. In some embodiments, the chemical functional groups (coupling partners) for a covalent coupling reaction are amine/carboxyl groups in an amide-bond forming reaction. In some embodiments, the coupling partners for a coupling reaction are thiol/maleimide groups in a Michael reaction. In some embodiments, the coupling partners for a coupling reaction are thiol/disulfide groups in a disulfide exchange reaction. In some embodiments, the coupling partners for a coupling reaction are hydroxyl/epoxy groups in an epoxy ring-opening reaction. In some embodiments, the coupling partners for a coupling reaction are amino/epoxy groups in an epoxy ring-opening reaction.

The chemical functional group is linked to the 5′ phosphate group of the oligonucleotides. In some embodiments, the chemical functional group is linked to the 5′ phosphate group of the oligonucleotides with a spacer of about 3 to about 16 carbon atoms, for example, a 3 carbon spacer, a 4 carbon spacer, a 5 carbon spacer, a 6 carbon spacer, a 7 carbon spacer, a 8 carbon spacer, a 9 carbon spacer, a 10 carbon spacer, a 11 carbon spacer, a 12 carbon spacer, a 13 carbon spacer, a 14 carbon spacer, a 15 carbon spacer, or a 16 carbon spacer. In some embodiments, the spacer is a 6 carbon spacer. In some embodiments, the spacer is a 12 carbon spacer.

### Methods for Using Alignment Beads in a Singleplexed or Multiplexed FISH Assay

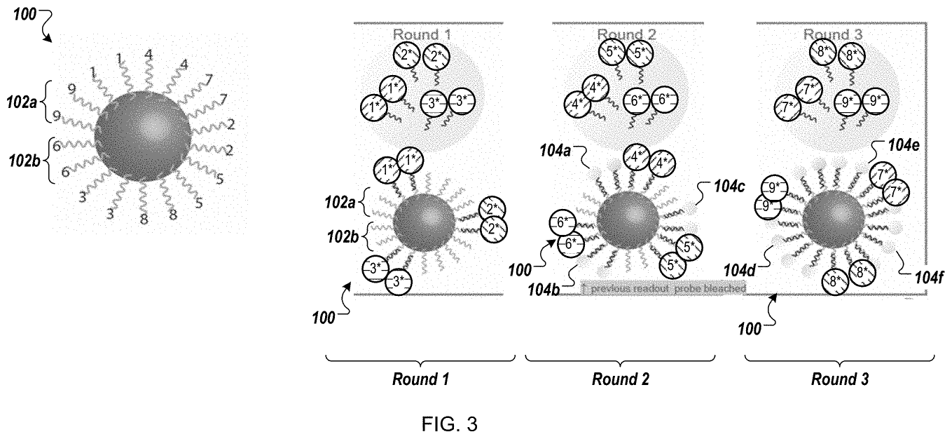

As described above, since the alignment beads (fiducial markers) can be functionalized with multiple different oligonucleotide binding sites, the alignment beads can be labeled with different readout-probes having with a single fluorophore (singleplex) or readout-probes having multiple different fluorophores (multiplex) for use in each round of hybridization and imaging. FIG. 3 depicts an alignment bead 100 functionalized with nine different binding sites 102a-b. The binding sites on the alignment bead encode reverse-complementary oligonucleotide sequences to the targeting domains of probes, which are conjugated to different color dyes. Three different probes are labeled with one dye (e.g., sequences 1, 4 and 7 are labeled with red dye, sequences 2, 5 and 8 are labeled with green dye, and sequences 3, 6 and 9 are labeled with yellow dye), and probes labeled in one or more colors can be used in a single hybridization round. For example, in Round 1, probes 1-3 are used; in Round 2, probes 4-6 are used; in Round 3, probes 7-9 are used. The sample is photo-bleached after imaging and registration in each round of hybridization, resulting on one or more bleached readout probes 104a-f during various rounds.

In one example of a single-color FISH experiment, six different single-color probes can be used to label the alignment beads in each round of hybridization. Alignment beads of 1-μm diameter and functionalized with oligonucleotide binding sites complementary to the six probes, each of a single color, can be used as fiducial markers. The same field of view can be imaged throughout the rounds of hybridization. Six rounds of hybridization can be performed, each round comprising (a) staining a sample with an aqueous buffer solution containing the alignment beads and DAPI; (b) hybridizing one type of single-color probes, each probe comprising an oligonucleotide targeting domain complementary to one oligonucleotide binding site of the alignment bead and a fluorescent dye; and (c) photo-bleaching the sample to remove the single-color probes at the end of each round. Under these conditions, the 1-μm alignment beads should show up in all rounds of imaging with different colors in each round. Offset of alignment beads between rounds can be used to correct for image registration. In general, a minimum of 3 beads per FOV should be sufficient to correct for shifts in the focus plane in between readout rounds.

An mFISH apparatus can correct for image registration between rounds by comparing two images of a sample, each captured during a different round. When mFISH apparatus determines that the alignment beads depicted in each of the two images are in approximately the same location within the images, e.g., are within a threshold distance of each other, the mFISH apparatus can determine to skip registration. When the mFISH apparatus determines that the locations of the alignment beads depicted in each of the two images are more than a threshold distance apart, the mFISH apparatus can determine a registration transformation for the image, e.g., a translation, rotation, or combination thereof, that that when applied to the image will move the location of each bead depicted in a first image to a corresponding second location of the bead depicted in a second image. The mFISH apparatus can then use apply this transformation to the image to perform image registration.

The mFISH apparatus can correct for registration by adjusting a location of a sample depicted in one of the images using the translation. This can cause the depiction of the sample in both images to be in approximately the same location.

Samples can also be imaged with alignment beads labeled with multiple-color probes in a single round of hybridization. In one example, fluorescent microscopy images can be acquired sequentially of a 1-μm diameter alignment bead having multiple copies of fifteen different oligonucleotide binding sites attached to the bead surface. Five rounds of hybridization can be performed, each round comprising (a) staining a sample with an aqueous buffer solution containing the alignment beads and DAPI; (b) hybridizing three types of single-color probes, each probe comprising an oligonucleotide targeting domain complementary to one oligonucleotide binding site of the alignment bead and a fluorescent dye (such as those described herein); and (c) photo-bleaching the sample to remove the single-color probes at the end of each round. Under these conditions, the 1-μm alignment beads should show up in all three fluorescence channels simultaneously during each round of hybridization and imaging. The tracking of three beads per FOV enables correction of shifts caused in between readout rounds. In another example, fluorescent microscopy images can be acquired sequentially of a 0.2-μm diameter alignment bead having multiple copies of eighteen different oligonucleotide binding sites attached to the bead surface. Six rounds of hybridization can be performed, each round comprising (a) staining a sample with an aqueous buffer solution containing the alignment beads and DAPI; (b) hybridizing three types of single-color probes, each probe comprising an oligonucleotide targeting domain complementary to one oligonucleotide binding site of the alignment bead and a fluorescent dye (such as those described herein); and (c) photo-bleaching the sample to remove the single-color probes at the end of each round. The 0.2-μm alignment beads should show up in all three fluorescence channels simultaneously during each round of hybridization and imaging. The tracking of three beads per FOV enables correction of shifts caused in between readout rounds.

### EXAMPLES

The following materials and methods were used in the Examples set forth herein.

### Example 1. Preparation of ssDNA

### PCR Amplification

DNA oligonucleotides with sequences for use in preparing functionalized alignment beads as fiducial markers to improve image registration in fluorescence assays are designed via a computational design step and synthesized by methods known in the field. The DNA oligonucleotides are then diluted to a working concentration of 0.4 ng/μL. Forward and Reverse PCR primers are designed and generated using methods known in the art. Forward and Reverse PCR primers are diluted to a working concentration of 100 μM. The PCR working solution is prepared according to Table 1:

TABLE 1

| Nuclease-Free Water (Ambion, # | 191.2 | uL |
|---|---|---|
| AM9932) | | |
| 100 uM Forward Primer | 2.0 | uL |
| 100 uM Reverse Primer | 2.0 | uL |
| Oligo Library | 0.4 | ng/uL |
| Phusion HotStart Flex 2X Master | 200 | uL |
| Mix (NEB, #M0536L) | | |
| Total volume | 500 | uL |

Volumes needed for each reagent for a total of 8 PCR reactions are mixed together. Specifically, 50 uL of the PCR working solution in Table 1 is added to each well in sterile (autoclaved) 8-strip PCR tubes 0.2 mL (e.g., Axygen, #14-222-250, or any equivalent) (“8-tube strips”). The 8-tube strips are vortexed gently (low setting) in a vortex mixer (e.g., Corning, cat no. #10-320-807, or any equivalent) and centrifuged in a centrifuge (Eppendorf, cat no. #022621431, or any equivalent) to ensure the PCR working solution is not stuck on the cover of the 8-tube strips and that there are no bubbles. The 8-tube strips are placed into the PCR thermocycler machine (ThermoFisher Scientific, SimpliAmp Thermal Cycler, or any equivalent) which is adjusted to carry out the PCR program in Table 2, which is designed for amplification of L1 oligo library.

TABLE 2

| | | | | | |
|---|---|---|---|---|---|
| 98.0° C. | 98.0° C. | 66.0° C. | 72.0° C. | 72.0° C. | 4.0° C. |

| | | |
|---|---|---|
| 1x | 25x | 1x |
| Stage 1 | Stage 2 | Stage 3 |

1 uL of PCR product is diluted in 10× with Nuclease Free Water and saved to run DNA ScreenTape analysis using methods known in the art (e.g., using Agilent D1000 ScreenTape Assay and Reagents Agilent, #5067-5582, #5067-5583, #5067-5586, following manufacturer instructions). A single, clean band should be seen, and this confirms that there was specific product amplification. The size of synthesized PCR product is also verified by the ScreenTape analysis. The concentration is also verified by the ScreenTape analysis based on peak graphs and the concentration should be around 10-50 ng/μl. If the PCR product is not used immediately it should be kept at 4° C. The samples are labeled with initials, library and date of amplification.

### PCR Product Ampure XP Beads Cleanup

To purify the PCR product, 90 μl of Ampure XP beads (Beckman, #A63882) is added into each well of 8-tube strips, mixed well, and incubated at room temperature for 5 minutes. The 8-tube strips are placed on a magnetic stand (e.g., DynaMag™-2 Magnet Magnetic Stand, ThermoFisher Scientific, #12321D) until the liquid is clear (e.g., after 5 minutes). This ensures that the Ampure XP beads are pulled down by magnetic force. The 8-tube strips are removed from the magnetic stand. 140 μl supernatant from each well is discarded. The Ampure XP beads are washed twice using the following protocol: a) adding 180 μl fresh 80% EtOH (ethanol) (Merck, #200-578-6) to each well; b) incubating on the magnetic stand for 30 seconds; c) removing and discarding all supernatant from each well. The Ampure XP beads are then air-dried on the magnetic stand for 15 minutes. The 8-tube strips are then removed from the magnetic stand. 11 μl Nuclease Free Water is added to each well of the 8-tube strips, mixed thoroughly, and then incubated at room temperature for 2 minutes. The 8-tube strips are placed on a magnetic stand until the liquid is clear (e.g., after 5 minutes). 11 μl supernatant from each well is transferred into a separate 1.5 ml Eppendorf tube (e.g., Axygen, #MCT-175-C, or any equivalent). 1 uL of the purified PCR product is diluted in 10× with Nuclease Free Water and saved to run DNA ScreenTape analysis using methods known in the art (e.g., using Agilent D1000 ScreenTape Assay and Reagents Agilent, #5067-5582, #5067-5583, #5067-5586, following manufacturer instructions).

### In Vitro Transcription (IVT)

The working solution for IVT is assembled following the recipe in Table 3 below:

TABLE 3

| | | |
|---|---|---|
| Purified PCR product from above | 80.0 | μL |
| NTP Buffer Mix | 128.0 | μL |
| RNasin Plus (40U/μL) | 8.0 | μL |
| T7 RNA Polymerase Mix, | 24.0 | μL |
| (HiScribe ™ Quick T7 High Yield RNA | | |
| Synthesis Kit, NEB, # E2050S) | | |

30 μl of the IVT working solution is added into each well of 8-tube strips. The 8-tube strips are then placed into a PCR thermocycler machine and incubated at 37° C. overnight. The IVT reaction is stopped by DNase treatment by adding 2 uL DNase (2 U/uL) and 20 uL Nuclease Free Water into each well of the 8-tube strips and incubating for 15 minutes. During this process, each well of the 8-tube strips should contain 30 uL of the IVT product from above, 20 uL Nuclease Free Water and 2 uL DNase (2 U/uL). After the DNase treatment, 0.5 μl of 0.5M EDTA (final concentration 5 mM) is added to each well of the 8-tube strips and incubated at 75° C. for 10 minutes.

### IVT Product RNA Beads Cleanup

To purify the IVT product, 94.5 μl of RNA beads (e.g., RNAClean XP from Beckman, Item No: A66514) is added into each well of the 8-tube strips, mixed well, and incubated at room temperature for 5 minutes. The 8-tube strips are placed on a magnetic stand until the liquid is clear (e.g., after 5 minutes). This ensures that the RNA beads are pulled down by magnetic force. The 8-tube strips are removed from the magnetic stand. 140 μl supernatant from each well is discarded. The RNA beads are washed twice using the following protocol: a) adding 180 μl fresh 80% EtOH (ethanol) to each well; b) incubating on the magnetic stand for 30 seconds; c) removing and discarding all supernatant from each well. The RNA beads are then air-dried on the magnetic stand for 15 minutes. The 8-tube strips are then removed from the magnetic stand. 11 μl Nuclease Free Water is added to each well of the 8-tube strips, mixed thoroughly, and then incubated at room temperature for 2 minutes. The 8-tube strips are placed on a magnetic stand until the liquid is clear (e.g., after 5 minutes). 11 μl supernatant from each well is transferred into a separate 1.5 ml Eppendorf tube (e.g., Axygen, #MCT-175-C, or any equivalent). 1 uL of the purified IVT product is diluted in 10× with Nuclease Free Water and saved to run RNA ScreenTape Analysis using methods known in the art (e.g., using Agilent TapeStation RNA ScreenTape & Reagents, #5067-5577, #5067-5578, #5067-5576, following manufacturer instructions). 1 uL of the purified IVT product is diluted in 10× with Nuclease Free Water and saved to run on a PAGE gel following methods known in the art.

### Reverse Transcription (RT)

The PCR thermocycler is pre-heated to 65° C. and kept ready for subsequent step. The working solution for RT is assembled following the recipe in Table 4 below:

TABLE 4

| Nuclease Free Water | 36.0 | μL |
|---|---|---|
| 25 mM dNTPs (100 mM each) (PCR | 20.0 | μL |
| Biosystems, # PB10.72-10) | | |
| 100 uM Forward Primer (15 nmol) | 150.0 | μL |
| IVT product | 80.0 | μL |
| 5x RT Buffer | 80.0 | μL |
| RNasin Plus (40 U/μL) (RNasin ® | 16.0 | μL |
| Plus RNase Inhibitor-10,000u, | | |
| Promega, # N2615) | | |
| Maxima H-Reverse Transcriptase | 18.0 | μL |

(Thermofisher, # EP0753)

After adding 36 μL Nuclease Free Water, 20 μL 25 mM dNTPs, 150 μL 100 uM Forward Primer and 80 μL IVT product, the working solution is heated at 65° C. for 5 min, and then put in ice immediately before adding the other components (5×RT buffer, RNasin Plus and Maxima H-Reverse Transcriptase). 50 μl of the RT working solution is added into each well of 8-tube strips and then incubated in the PCR thermocycling machine at 50° C. for 1 hour.

The RT reaction is stopped by alkaline hydrolysis by adding 10 μL 0.5 M EDTA and 10 μL 1 M NaOH into each well of the 8-tube strips. For the alkaline hydrolysis step, the 8-tube strips are incubated at 65° C. for 15 min and cooled on ice for 5 minutes. 9.8 μL of 2 M HCl is added to each well of the 8-tube strips to step the alkaline hydrolysis. After adding HCl, the pH of the solution should be around pH 7-9.

### RT Product Ampure XP Beads Cleanup

To purify the PCR product, 145 μl of Ampure XP beads (Beckman, #A63882) is added into each well of 8-tube strips, mixed well, and incubated at room temperature for 5 minutes. The 8-tube strips are placed on a magnetic stand (e.g., DynaMag™-2 Magnet Magnetic Stand, ThermoFisher Scientific, #12321D) until the liquid is clear (e.g., after 5 minutes). This ensures that the Ampure XP beads are pulled down by magnetic force. The 8-tube strips are removed from the magnetic stand. 140 μl supernatant from each well is discarded. The Ampure XP beads are washed twice using the following protocol: a) adding 180 μl fresh 80% EtOH (ethanol) (Merck, #200-578-6) to each well; b) incubating on the magnetic stand for 30 seconds; c) removing and discarding all supernatant from each well. The Ampure XP beads are then air-dried on the magnetic stand for 15 minutes. The beads should not be over-dried. The 8-tube strips are then removed from the magnetic stand. 11 μl Nuclease Free Water is added to each well of the 8-tube strips, mixed thoroughly, and then incubated at room temperature for 2 minutes. The 8-tube strips are placed on a magnetic stand until the liquid is clear (e.g., after 5 minutes). 11 μl supernatant from each well is transferred into a separate 1.5 ml Eppendorf tube (e.g., Axygen, #MCT-175-C, or any equivalent). 1 uL of the purified RT product is diluted in 10× with Nuclease Free Water and saved to run DNA ScreenTape analysis using methods known in the art (e.g., using Agilent D1000 ScreenTape Assay and Reagents Agilent, #5067-5582, #5067-5583, #5067-5586, following manufacturer instructions). The size and concentration of the purified RT product are checked by DNA ScreenTape analysis. Here, the concentration of the purified RT product should be around 2000-5000 ng/μl. The purified RT product is stored at −20° C.

### Example 2. Functionalization of 0.2-μm Carboxyl Latex Beads with 5′-Amino-Oligonucleotides

Following Example 1, the ssDNA sequences are designed for use in preparing functionalized alignment beads and prepared using the PCR-IVT-RT method. In this example, the ssDNA is a mixture of 16 difference sequences. In order to functionalize the alignment beads, an amino group is covalently linked to the 5′-phosphate group of the ssDNA with a 12-carbon spacer in between. This can be achieved using a commercial amino linker C12 (e.g., GeneLink Cat. No. 26-6420), following manufacturer instructions. 40 μL of 1 mM (corresponding to 2.5 nmol) of 5′-amino oligonucleotide solution was transferred to a 1.8-mL conical tube via an automatic micropipette. To the oligonucleotides solution was added 1 volume of MES buffer. The resulting solution was vortexed gently to mix the components.

1 pmol of a 4% (w/v) aqueous suspension of the 0.2-μm diameter carboxyl latex beads (Catalog no. C37486, Molecular Probes) was transferred to a 1.8-mL conical tube via a micropipette, to which 3 volumes of MES buffer was added, resulting in a 1% (w/v) bead suspension. The bead suspension was centrifuged at 12,000 g for 15 min to fully precipitate the beads. The supernatant was discarded, and the pellet was re-suspended in 400-μL of MES buffer with gentle vortexing. The resulting bead suspension was added to the oligonucleotide/MES solution prepared as described above.

A stock solution of 10 mg/mL EDC in ultrapure water was prepared and 5-μL (corresponding to about 250 nmol of EDC) of the stock solution was added to the bead/oligonucleotide mixture as described above to initiate the coupling reaction. The resulting mixture was incubated at room temperature for 3 hours, and then terminated by adding 50-4, of 1M Tris buffer to the reaction mixture followed by vortexing for 15 min. The mixture was then centrifuged at 12,000 g for 15 min, upon which the supernatant was discarded and the pellet re-suspended in 500-μL of TE buffer. The centrifugation/re-suspension steps were repeated once and the resulting oligonucleotide-functionalized beads were stored at refrigerated temperature (2-8° C.) until used.

### Example 3. Oligonucleotide Alignment Beads in a FISH Imaging Experiment

A 5-μL aliquot of the oligonucleotide-functionalized alignment beads, as prepared in Example 2, was mixed with 1 mL of each of a 1-10 μg/mL DAPI solution in PBS (in 1 μg/mL increments), to target a 0.5-1% (v/v) bead/DAPI ratio. The resulting mixture was sonicated for 10 min, applied to a microscope slide. After 15 min, the sample was washed twice with 2×SSC, post-fixed with 4% PFA in PBS for 15 min, and then washed 3 times with 2×SSC. The sample containing alignment beads were subsequently imaged with the mFISH apparatus and the image capture process, described in more detail below.

### FISH Imaging System

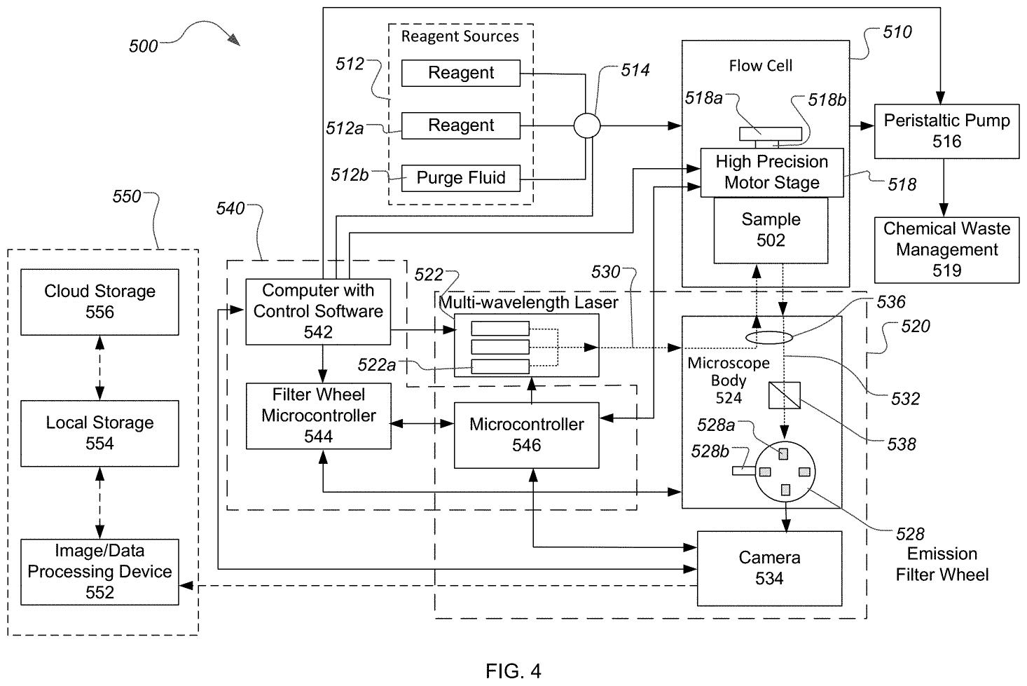

Referring to FIG. 4, a multiplexed fluorescent in-situ hybridization (mFISH) imaging and image processing apparatus 500 includes a flow cell 510 to hold a sample 502, a fluorescence microscope 520 to obtain images of the sample 502, and a control system 540 to control operation of the various components of the mFISH imaging and image processing apparatus 500. The control system 540 can include a computer 542, e.g., having a memory, processor, etc., that executes control software.

The fluorescence microscope 520 includes an excitation light source 522 that can generate excitation light 530 of multiple different wavelengths. In particular, the excitation light source 522 can generate narrow-bandwidth light beams having different wavelengths at different times. For example, the excitation light source 522 can be provided by a multi-wavelength continuous wave laser system, e.g., multiple laser modules 522a that can be independently activated to generate laser beams of different wavelengths. Output from the laser modules 522a can be multiplexed into a common light beam path.

The fluorescence microscope 520 includes a microscope body 524 that includes the various optical components to direct the excitation light from the light source 522 to the flow cell 510. For example, excitation light from the light source 522 can be coupled into a multimode fiber, refocused and expanded by a set of lenses, then directed into the sample 502 by a core imaging component, such as a high numerical aperture (NA) objective lens 536. When the excitation channel needs to be switched, one of the multiple laser modules 522a can be deactivated and another laser module 522a can be activated, with synchronization among the devices accomplished by one or more microcontrollers 544, 546.

The objective lens 536, or the entire microscope body 524, can be installed on vertically movable mount coupled to a Z-drive actuator. Adjustment of the Z-position, e.g., by a microcontroller 546 controlling the Z-drive actuator, can enable fine tuning of focal position. Alternatively, or in addition, the flow cell 510 (or a stage 518 supporting the sample in the flow cell 510) could be vertically movable by a Z-drive actuator 518b, e.g., an axial piezo stage. Such a piezo stage can permit precise and swift multi-plane image acquisition.

The sample 502 to be imaged is positioned in the flow cell 510. The flow cell 510 can be a chamber with cross-sectional area (parallel to the object or image plane of the microscope) with and area of about 2 cm by 2 cm. The sample 502 can be supported on a stage 518 within the flow cell, and the stage (or the entire flow cell) can be laterally movable, e.g., by a pair of linear actuators 518a to permit XY motion. This permits acquisition of images of the sample 502 in different laterally offset fields of view (FOVs). Alternatively, the microscope body 524 could be carried on a laterally movable stage.

An entrance to the flow cell 510 is connected to a set of hybridization reagents sources 512. A multi-valve positioner 514 can be controlled by the controller 540 to switch between sources to select which reagent 512a is supplied to the flow cell 510. Each reagent includes a different set of one or more oligonucleotide probes, e.g., readout probes. Each probe targets a different RNA sequence of interest, and has a different set of one or more fluorescent materials, e.g., phosphors, that are excited by different combinations of wavelengths. In addition to the reagents 512a, there can be a source of a purge fluid 512b, e.g., deionized (“DI”) water.

An exit to the flow cell 510 is connected to a pump 516, e.g., a peristaltic pump, which is also controlled by the controller 540 to control flow of liquid, e.g., the reagent or purge fluid, through the flow cell 510. Used solution from the flow cell 510 can be passed by the pump 516 to a chemical waste management subsystem 519.

In operation, the controller 540 causes the light source 522 to emit the excitation light 530, which causes fluorescence of fluorescent material in the sample 502, e.g., fluorescence of the probes that are bound to RNA in the sample and that are excited by the wavelength of the excitation light. The emitted fluorescent light 532, as well as back propagating excitation light, e.g., excitation light scattered from the sample, stage, etc., is collected by an objective lens 536 of the microscope body 524.

The collected light can be filtered by a multi-band dichroic mirror 538 in the microscope body 524 to separate the emitted fluorescent light from the back propagating illumination light, and the emitted fluorescent light is passed to a camera 534. The multi-band dichroic mirror 538 can include a pass band for each emission wavelength expected from the probes, e.g., the readout probes, under the variety of excitation wavelengths. Use of a single multi-band dichroic mirror (as compared to multiple dichroic mirrors or a movable dichroic mirror) can provide improved system stability.

The camera 534 can be a high resolution (e.g., 2048×2048 pixel) CMOS (e.g., a scientific CMOS) camera, and can be installed at the immediate image plane of the objective. Other camera types, e.g., CCD, may be possible. When triggered by a signal, e.g., from a microcontroller, image data from the camera can be captured, e.g., sent to an image processing system 550. Thus, the camera 534 can collect a sequence of images from the sample.

To further remove residual excitation light and minimize cross talk between excitation channels, each laser emission wavelength can be paired with a corresponding band-pass emission filter 528a. Each filter 528a can have a wavelength of 10-50 nm, e.g., 14-32 nm. In some implementations, a filter is narrower than the bandwidth of the fluorescent material of the probe resulting from the excitation, e.g., if the fluorescent material of the probe has a long trailing spectral profile.

The filters are installed on a high-speed filter wheel 528 that is rotatable by an actuator. The filter wheel 528 can be installed at the infinity space to minimize optical aberration in the imaging path. After passing the emission filter of the filter wheel 528, the cleaned fluorescence signals can be refocused by a tube lens and captured by the camera 534. The dichroic mirror 538 can be positioned in the light path between the objective lens 538 and the filter wheel 528.

To facilitate high speed, synchronized operation of the system, the control system 540 can include two microcontrollers 544, 546 that are employed to send trigger signals, e.g., TTL signals, to the components of the fluorescence microscope 520 in a coordinated manner. The first microcontroller 544 is directly run by the computer 542, and triggers actuator 528b of the filter wheel 528 to switch emission filters 528a at different color channels. The first microcontroller 544 or the computer 542 can trigger the second microcontroller 546, which sends digital signals to the light source 522 in order to control which wavelength of light is passed to the sample 502. For example, the second microcontroller 546 can send on/off signals to the individual laser modules of the light source 522 to control which laser module is active, and thus control which wavelength of light is used for the excitation light. After completion of switching to a new excitation channel, the second microcontroller 546 controls the motor for the piezo stage 518b to select the imaging height. Finally the second microcontroller 546 sends a trigger signal to the camera 534 for image acquisition.

Communication between the computer 542 and the device components of the mFISH apparatus 500 is coordinated by the control software. This control software can integrate drivers of all the device components into a single framework, and thus can allow a user to operate the imaging system as a single instrument (instead of having to separately control many devices).

The control software supports interactive operations of the microscope and instant visualization of imaging results. In addition, the control software can provide a programming interface which allows users to design and automate their imaging workflow. A set of default workflow scripts can be designated in the scripting language.

In some implementations, the control system 540 is configured, i.e., by the control software and/or the workflow script, to acquire fluorescence images (also termed simply “collected images” or simply “images”) in loops in the following order (from innermost loop to outermost loop): z-axis, color channel, lateral position, and reagent.

These loops may be represented by the pseudocode in Table 5, below.

TABLE 5
example control system loop pseudocode

```
for h = 1:N_hybridization
  % multiple hybridizations
  for f = 1:N_FOVs
    % multiple lateral field-of-views
    for c = 1:N_channels
      % multiple color channels
      for z = 1:N_planes
        % multiple z planes
        Acquire image(h, f, c, z);
      end % end for z
    end % end for c
  end % end for f
end % end for h
```

For the z-axis loop, the control system 540 causes the stage 518 to step through multiple vertical positions. Because the vertical position of the stage 518 is controlled by a piezoelectric actuator, the time required to adjust positions is small and each step in this loop can be extremely fast.

First, the sample can be sufficiently thick, e.g., a few microns, that multiple image planes through the sample may be desirable. For example, multiple layers of cells can be present, or even within a cell there may be a vertical variation in gene expression. Moreover, for thin samples, the vertical position of the focal plane may not be known in advance, e.g., due to thermal drift. In addition, the sample 502 may vertically drift within the flow cell 510. Imaging at multiple Z-axis positions can ensure most of the cells in a thick sample are covered, and can help identify the best focal position in a thin sample.

For the color channel loop, the control system 540 causes the light source 522 to step through different wavelengths of excitation light. For example, one of the laser modules is activated, the other laser modules are deactivated, and the emission filter wheel 528 is rotated to bring the appropriate filter into the optical path of the light between the sample 502 and the camera 534.

For the lateral position, the control system 540 causes the light source 522 to step through different lateral positions in order to obtain different fields of view (FOVs) of the sample. For example, at each step of the loop, the linear actuators supporting the stage 518 can be driven to shift the stage laterally. In some implementations, the control system 540 number of steps and lateral motion is selected such that the accumulated FOVs to cover the entire sample 502. In some implementations, the lateral motion is selected such that FOVs partially overlap.

For the reagent, the control system 540 causes the mFISH apparatus 500 to step through multiple different available reagents. For example, at each step of the loop, the control system 540 can control the valve 514 to connect the flow cell 510 to the purge fluid 512b, cause the pump 516 to draw the purge fluid through the cell for a first period of time to purge the current reagent, then control the valve 514 to connect the flow cell 510 to different new reagent, and then draw the new reagent through the cell for a second period of time sufficient for the probes in the new reagent to bind to the appropriate RNA sequences. As a result, a fluorescence image is acquired for each combination of possible values for the z-axis, color channel (excitation wavelength), lateral FOV, and reagent.

A data processing system 550 is used to process the images and determine gene expression to generate the spatial transcriptomic data. At a minimum, the data processing system 550 includes a data processing device 552, e.g., one or more processors controlled by software stored on a computer readable medium, and a local storage device 554, e.g., non-volatile computer readable media, that receives the images acquired by the camera 534. For example, the data processing device 552 can be a work station with GPU processors or FPGA boards installed. The data processing system 550 can also be connected through a network to remote storage 556, e.g., through the Internet to cloud storage.

The data processing system 550 can process the images as described in more detail below. For instance, the data processing system 550 can perform one or more steps to stich images from different FOVs together.

In some implementations, the data processing system 550 performs on-the-fly image processing as the images are received. In particular, while data acquisition is in progress, the data processing device 552 can perform image pre-processing steps, such as filtering and deconvolution, that can be performed on the image data in the storage device 554 but which do not require the entire data set. Because filtering and deconvolution can be a major bottleneck in the data processing pipeline, pre-processing as image acquisition is occurring can significantly shorten the offline processing time and thus improve the throughput.

### Image Capture Process

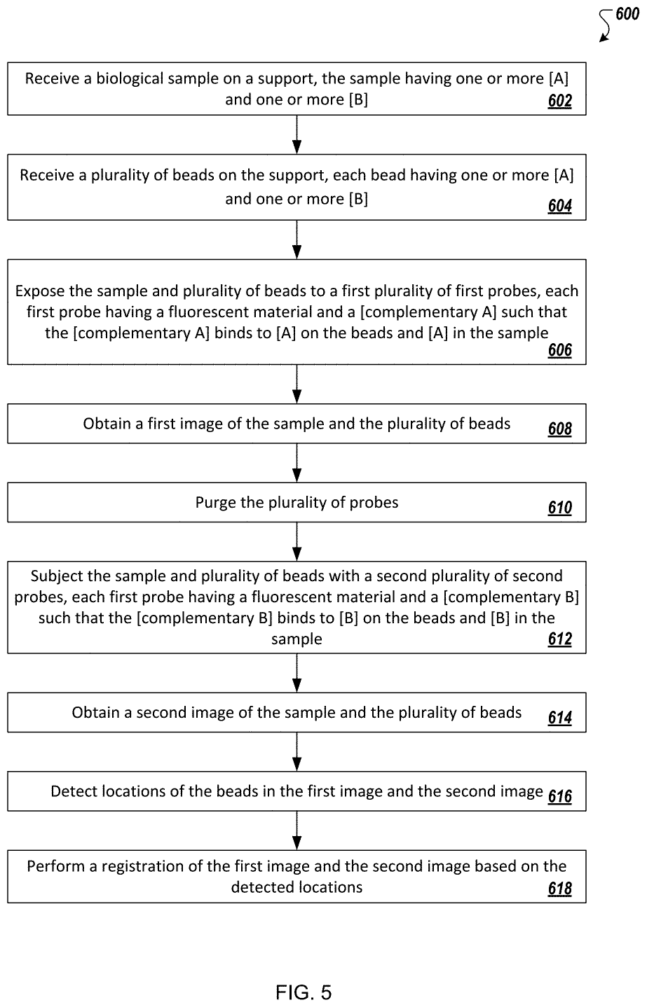

FIG. 5 is a flow diagram of a process 600 for registering a first image and a second image. For example, the process 600 can be used by the mFISH apparatus 500, e.g., an mFISH system, described with reference to FIG. 4.

An mFISH system receives a biological sample on a support (602). An operator can place the sample on the support in the mFISH system. For example, mFISH system receives the sample 502 on the flow cell 510, as described above. The sample, e.g., as part of RNA or DNA, (or a first targeting probe, e.g., as part of a readout portion of the targeting probe, that binds to the sample) has a first nucleotide sequence. The sample, e.g., as part of RNA or DNA, (or a second targeting probe, e.g., as part of a readout portion of the second targeting probe, that binds to the sample) has a second nucleotide sequence.

The mFISH system receives a plurality of beads on the support (604). Each bead has a plurality of binding sites. A first subset of the plurality of binding sites include the first nucleotide sequence. A second subset of the plurality of binding sites include the second nucleotide sequence. The beads can be fiducial markers as described in more detail above. For instance, each bead in the plurality of beads can be an alignment bead such as the alignment bead 100 described with reference to FIG. 1.

The mFISH system exposes the sample and plurality of beads to a first plurality of first probes (606). Each first probe has a fluorescent material and a complementary first nucleotide sequence such that the complementary first nucleotide sequence binds to the first nucleotide sequence on the beads and the first nucleotide sequence in the sample or in the targeting probe. For example, the mFISH system can expose the sample and the plurality of beads to the first plurality of first readout probes. The probes can be readout probes such as those described in more detail above.

In some examples, each of the first probes binds to either one of the beads or the sample. In some implementations, only a subset of the first probes binds to one of the beads or the sample. In these implementations, some of the first probes might not bind to a bead or the sample.

The mFISH system obtains a first image of the sample and the plurality of beads (608). For instance, the mFISH system positions the sample, and the support, at a first location and obtains the first image using a camera as described in more detail above. The mFISH system then obtains the first image that depicts both the sample and the plurality of beads. The first image can have a first z-axis location, a first color channel, and a first lateral position. The first lateral position can include a first x-axis location, and a first y-axis location.

The mFISH system can obtain different images using different color channels, as described in more detail above. For instance, the mFISH system can obtain the first image at a first color channel from multiple color channels and a second image at a second different color channel.

As part of the process to obtain an image, the mFISH system can excite fluorophores in the probes. The mFISH system can excite a fluorophore by sending a signal, generated by an excitation source such as a fluorescent microscopy apparatus, into the fluorophore that excites the fluorophore and causes the fluorophore to emit a fluorescent signal. The mFISH system can use a camera to obtain an image that depicts the fluorescent signals emitted by the fluorophores in the first probes.

The first image can depict data for all or a subset of the plurality of beads, all or a portion of the sample, or both. For instance, the first image can depict data for two or three of the beads and a portion of the sample.

The mFISH system purges the plurality of probes (610). For example, the mFISH system can use a purge fluid to purge the plurality of probes as described above. The purge process can purge all probes in the plurality of probes from the support. In some examples, the purge process removes some of the probes in the plurality of probes from the support, but not all probes in the plurality of probes. This can occur when the purge process removes probes from the support that have not bound to one of the beads or to the sample.

In some implementations, instead of or as part of the purge process, the mFISH system can photobleach the sample, the beads, the probes, or a combination of two or more of these. For instance, the mFISH system can photobleach the sample, as described in more detail above. Photobleaching the sample can also cause photobleaching of the beads, the probes, or both, that are on the support.

The mFISH system subjects the sample and plurality of beads to a second plurality of second probes. Each second probe has a fluorescent material and a complementary second nucleotide sequence such that the complementary second nucleotide sequence binds to the second nucleotide sequence on the beads and the second nucleotide sequence in the sample or in the second targeting probes (612). The second probes are different probes from the first probes. The first plurality and the second plurality can be the same quantity or different quantities. The second probes can be readout probes, such as readout probes described in more detail above.

In some examples, each of the second probes binds to either one of the beads or the sample. In some implementations, only a subset of the second probes binds to one of the beads or the sample. In these implementations, some of the second probes might not bind to a bead or the sample.

The mFISH system obtains a second image of the sample and the plurality of beads (614). For example, the mFISH system uses the camera to obtain the second image. The mFISH system obtain the second image that depicts both the sample and the plurality of beads. The second image can depict data for all or a subset of the plurality of beads, all or a portion of the sample, or both. For instance, the second image can depict data for two or three of the beads and a portion of the sample.

The mFISH system obtain the second image using a second color channel. The second color channel can be the same color channel as the first color channel. The second color channel can be a different color channel as the first color channel.

The second image has a second z-axis location, and a second lateral position. The second lateral position has a second x-axis location and a second y-axis location. The second z-axis location can be the same z-axis location as the first z-axis location.

In some examples, when the lateral position is two-dimensional, the first lateral position and the second lateral position can overlap partially, e.g., for a stitching process, or completely, e.g., for a registration process. In these examples, the first image and the second image can both depict data for at least one of the plurality of beads. For example, the first image and the second image can both depict data for a first bead. The first image can depict data for two other beads that are not depicted in the second image. The second image can depict data for three other beads that are not depicted in the first image.

Both the first image and the second image can each depict different portions of the sample. The different portions of the sample can be overlapping while not being the exact same portion of the sample.

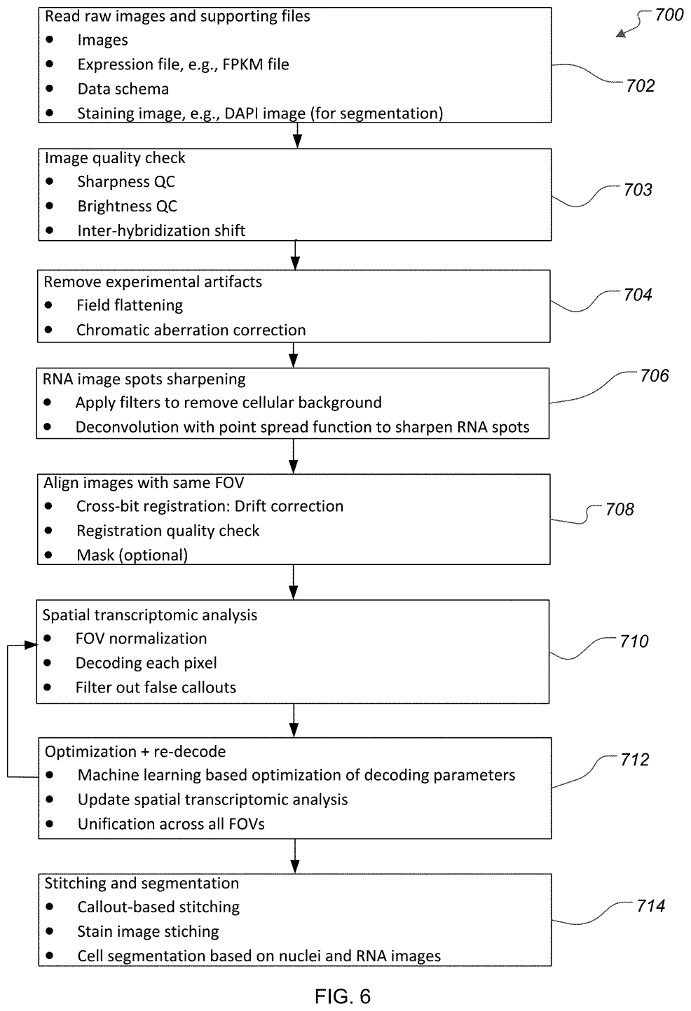

The mFISH system detects locations of the beads in the first image and the second image (616). For example, the mFISH system can detect the locations of the beads as described in more detail above and with reference to step 708 in FIG. 6.

Although the first image and the second image were captured at different lateral positions, and with different probes bound to the first bead, the mFISH system can detect the location of the first bead using data from the first image and the second image that represents the probes attached to the first bead. The mFISH system can determine that the first bead is depicted in both the first image and the second image using any appropriate process. For instance, the mFISH system can determine features depicted in each of the two images. The mFISH system can use properties of the depicted first bead, and features near the first bead that are depicted in both the first image and the second image, to determine that both images depict the same bead.

The mFISH system performs a registration of the first image and the second image based on the detected locations (618). For instance, the mFISH system can register the first image and the second image as described in more detail below, e.g., with reference to step 708 in FIG. 6 below.

The order of steps in the process 600 described above is illustrative only, and registering the first image and the second image can be performed in different orders. For example, the mFISH system can provide the plurality of beads on the support and then provide the biological sample on the support.

In some implementations, the process 600 can include additional steps, fewer steps, or some of the steps can be divided into multiple steps. For example, the mFISH system can perform one or more steps from the process 700 described with reference to FIG. 6 below.

### Image Stitching Process

FIG. 6 illustrates a flow chart of a process 700 of data processing in which the processing is performed after all of the images have been acquired. Although the process 700 is described as being performed after all images have been acquired, one or more steps in the process 700 can be performed before all images have been acquired. For instance, step 703, step 704, step 706, or a combination of these steps, can be performed for one or more first images while a data processing system, e.g., an image processing apparatus, continues to acquire one or more second images.

The process 700 begins with a data processing system receiving the raw image files and supporting files (step 702). In particular, the data processing system can receive the full set of raw images from the camera, e.g., an image for each combination of possible values for the z-axis, color channel (excitation wavelength), lateral FOV, and reagent.

The collected images can be subjected to one or more quality metrics (step 703) before more intensive processing in order to screen out images of insufficient quality. Depending on parameters for the data processing system, only images that meet the quality metric(s) can be passed on for further processing. This can significantly reduce processing load on the data processing system. For example, a sharpness quality value can be determined for each collected image to detect focusing failures As another example, in order to detect regions of interest, a brightness quality value can be determined for each collected image.

Next, some of the images can be processed to remove experimental artifacts (step 704). Since each RNA molecule will be hybridized multiple times with probes at different excitation channels, a strict alignment across the multi-channel, multi-round image stack can be beneficial for revealing RNA identities over the whole FOV. Removing the experimental artifacts can include field flattening and/or chromatic aberration correction. In some implementations, the field flattening is performed before the chromatic aberration correction.

One or more of the images can be processed to provide RNA image spot sharpening (step 706). RNA image spot sharpening can include applying filters to remove cellular background and/or deconvolution with point spread function to sharpen RNA spots.

The images having the same FOV are registered to align the features, e.g., the cells or cell organelles, therein (step 708). To accurately identify RNA species in the image sequences, features in different rounds of images are aligned, e.g., to sub-pixel precision. The images from the different rounds of images can each have a different color channel. For instance, one image can be captured at a first color channel for a first fluorescent material for the first probes used during that first round of imaging while another image can be captured at a second different color channel for a second different fluorescent material for the second probes used during that second round of imaging.

However, since an mFISH sample is imaged in aqueous phase and moved around by a motorized stage, sample drifts and stage drifts through hours-long imaging process can transform into image feature shifts, which can undermine the transcriptomic analysis if left unaddressed. In other words, even assuming precise repeatable alignment of the fluorescence microscope to the flow cell or support, the sample may no longer be in the same location in the later image, which can introduce errors into decoding or simply make decoding impossible.

The data processing apparatus can register images by placing fiducial markers, e.g., fluorescent beads, within the carrier material on the slide. In general, the sample and the fiducial marker beads will move approximately in unison. The data processing apparatus can identify these beads in the image based on their size and shape. Comparison of the positions of the beads can enable the data processing apparatus to register the two images, e.g., calculate of an affine transformation between the two images.

As part of this process, the data processing apparatus can use features for the beads, for the portions of the sample surrounding the beads, or both, to detect beads that are depicted in multiple images. The multiple images can be images captured during different processing rounds. The data processing apparatus can then use these features to determine the images that depict the same bead. The data processing apparatus can then register the images that depict the same bead, e.g., as described in more detail above.

Optionally, after registration, a mask can be calculated for each collected image. In brief, the intensity value for each pixel is compared to a threshold value. A corresponding pixel in the mask is set to 1 if the intensity value is above the threshold, and set to 0 if the intensity value is below the threshold. The threshold value can be an empirically determined value, e.g., predetermined value, or can be calculated from the intensity values in the image. In general, the mask can correspond to the location of cells within the sample; spaces between cells should not fluoresce and should have a low intensity.

After registration of the images in a FOV, spatial transcriptomic analysis can be performed (step 710).

FOV normalization can be performed before the spatial transcriptomic analysis in order to make the histogram more consistent. In some implementations, the FOV normalization occurs after registration. Alternatively, FOV normalization can occur before registration. FOV normalization could be considered part of the filtering.

After normalization, an image stack can be evaluated as a 2-D matrix of pixel words as part of a process to decode each pixel. The matrix can have P rows, where P=X*Y, and B columns, where B is the number of images in the stack for a given FOV, e.g., N_hybridization*N_channels. Each row corresponds to one of the pixels (the same pixel across the multiple images in the stack), the values from the row provide a pixel word. Each column provides one of the values in the word, i.e., the intensity value from the image layer for that pixel. The values can be normalized, e.g., vary between 0 and I_{MAX}. I_{MAX} can have a value of 1.

If all the pixels are passed to the decoding step, then all P words will be processed as described below. However, pixels outside cell boundaries can be screened out by the 2-D masks and not processed. As result, computational load can be significantly reduced in the following analysis.

The data processing system 550 can store a code book that is used to decode the image data to identify the gene expressed at the particular pixel. The code book can include multiple reference code words, each reference code word associated with a particular gene. The code book can be represented as a 2D matrix with G rows, where G is the number of code words, e.g., the number of genes (although the same gene could be represented by multiple code words), and B columns. Each row can correspond to one of the reference code words, and each column can provide one of the values in the reference code word, as established by prior calibration and testing of known genes. For each column, the values in the reference code can be binary, i.e., “on” or “off”. For example, each value can be either 0 or I_{MAX}, e.g., 1.

For each pixel to be decoded, a distance d(p,i) is calculated between the pixel word and each reference code word. For example, the distance between the pixel word and reference code word can be calculated as a Euclidean distance, e.g., a sum of squared differences between each value in the pixel word and the corresponding value in the reference code word. This calculation can be expressed as:

$$d ( p , i ) = {\sum }_{x = 1}^{B} {( {I}_{p , x} - {C}_{i , x} )}^{2}$$

where I_{p,x} are the values from the matrix of pixel words and C_{i,x} are the values from the matrix of reference code words. Other metrics, e.g., sum of absolute value of differences, cosine angle, correlation, etc., can be used instead of a Euclidean distance.

Once the distance values for each code word are calculated for a given pixel, the smallest distance value is determined, the code word that provides that smallest distance value is selected as the best matching code word. Stated differently, the data processing apparatus determines min (d(p,1), d(p,2), . . . d(p,B)), and determines the value b as the value for i (between 1 and B) that provided the minimum. The gene corresponding to that best matching code word is determined, e.g., from a lookup table that associates code words with genes, and the pixel is tagged as expressing the gene.

The data processing apparatus can filter out false callouts. One technique to filter out false callouts is to discard tags where the distance value d(p,b) that indicated expression of a gene is greater than a threshold value, e.g., if d(p,b)>D1_{MAX}.

Yet another technique for filtering false callouts is to reject code words where a calculated bit ratio BR falls below a threshold. The bit ratio is calculated as the mean of the intensity values from the image word for layers that are supposed to be on (as determined from the code word), divided by the mean of the intensity values from the image word for layers that are supposed to be off (again as determined from the code word).

The bit ratio BR is compared to a threshold value TH_{BR}. In some implementations, the threshold value TH_{BR} is determined empirically from prior measurements. However, in some implementations, the threshold value TH_{BR} can be calculated automatically for a particular code word based on the measurements obtained from the sample.

Yet another technique for filtering false callouts is to reject code words where a calculated bit brightness BB falls below a threshold. The bit brightness is calculated as the mean of the intensity values from the image word for layers that are supposed to be on (as determined from the code word).

The bit brightness BB is compared to a threshold value TH_{BB}. In some implementations, the threshold value TH_{BB} is determined empirically from prior measurements. However, in some implementations, the threshold value TH_{BB} can be calculated automatically for a particular code word based on the measurements obtained from the sample.

The data processing apparatus can perform optimization and re-decoding (step 712). The optimization can include machine-learning based optimization of the decoding parameters, followed by updating spatial transcriptomic analysis using updated decoding parameters. This cycle can be repeated until the decoding parameters have stabilized.

The optimization of the decoding parameters can use a merit function, e.g., a FPKM/TPM correlation, spatial correlation, or confidence ratio. Parameters that can be included as variables in the merit function include the shape (e.g., start and end of frequency range, etc.) of the filters used to remove cellular background, the numerical aperture value for the point spread function used to sharpen the RNA spots, the quantile boundary Q used in normalization of the FOV, the bit ratio threshold TH_{BR}, the bit brightness threshold TH_{BB} (or the quantiles used to determine the bit ratio threshold TH_{BR} and bit brightness threshold TH_{BB}), and/or the maximum distance D1ₘₐₓ at which at which a pixel word can be considered to match a code word.

This merit function may be an effectively discontinuous function, so a conventional gradient following algorithm may be insufficient to identify the optimal parameter values. A machine learning model can be used to converge on parameter values.

Next, the data processing apparatus can perform unification of the parameter values across all FOVs. Because each FOV is processed individually, each field can experience different normalization, thresholding, filtering setting, or a combination of two or more of these. As a result, a high contrast image can result in a histogram with variation that causes false positive callouts in quiet areas. The result of unification is that all FOVs use the same parameter values. This can significantly remove callouts from background noise in quiet area, and can provide a clear and unbiased spatial pattern in large sample area.

A variety of approaches are possible to select a parameter value that will be used across all FOVs. One option is to simply pick a predetermined FOV, e.g., the first measured FOV or a FOV near the center of the sample, and use the parameter value for that predetermined FOV. Another option is to average the values for the parameter across multiple FOV and then use the averaged value. Another option is to determine which FOV resulted in the best fit between its pixel words and tagged code words. For example, a FOV with the smallest average distance d(p,b1) between the tagged code words and the pixel words for those code words can be determined and then selected.

The data processing apparatus can perform stitching and segmentation (step 714). Stitching combines multiple FOVs into a single image. Stitching can be performed using a variety of techniques. One approach is, for each row of FOV that together will form the combined image of the sample and each FOV within the row, determine a horizontal shift for each FOV. Once the horizontal shifting is calculated, a vertical shift is calculated for each row of FOV. The horizontal and vertical shifts can be calculated based on cross-correlation, e.g., phase correlation. With the horizontal and vertical shift for each FOV, a single combined image can be generated, and gene coordinates can be transferred to the combined image based on the horizontal and vertical shift.

An indication that a gene is expressed at a certain coordinate in the combined fluorescence image (as determined from the coordinate in the FOV and the horizontal and vertical shift for that FOV) can be added, e.g., as metadata. This indication can be termed a “callout.”

The stain images, e.g., the DAPI images, can be stitched together to generate a combined stain image. In some implementations, it is not necessary to create a combined fluorescence image from the collected fluorescence images; once the horizontal and vertical shift for each FOV is determined, the gene coordinates within the combined stain image can be calculated. The stain image can be registered to the collected fluorescent image(s). An indication that a gene is expressed at a certain coordinate in the combined stain image (as determined from the coordinate in the FOV and the horizontal and vertical shift for that FOV) can be added, e.g., as metadata, to provide a callout.

A potential problem remains in the stitched image. In particular, some genes may be double-counted in the overlapping area. To remove double-counting, a distance, e.g., Euclidean distance, can be calculated between each pixel tagged as expressing a gene and other nearby pixels tagged as expressing the same gene. One of the callouts can be removed if the distance is below a threshold value. More complex techniques can be used if a cluster of pixels are tagged as expressing a gene.

Segmentation of the combined image, e.g., the image of the stained cell, into regions corresponding to cells can be performed using various known techniques. Segmentation is typically performed after stitching of the images, but can occur before or after callouts are added to the combined image.

The segmented image with callouts indicating positions of gene expression, can now be stored and presented to a user, e.g., on a visual display, for analysis.

Although the discussion above assumes that a single z-axis image is used in for each FOV, this is not required. Images from different z-axis positions can be processed separately; effectively the different z-axis positions provide a new set of FOVs.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method of generating single-stranded DNA (ssDNA), the method comprising:
(a) providing a plurality of DNA oligonucleotides;
(b) performing a polymerase chain reaction (PCR) amplification of the DNA oligonucleotides;
(c) purifying the PCR products with magnetic beads;
(d) performing in vitro transcription (IVT) reaction with the PCR products to generate intermediate RNA molecules;
(e) performing reverse transcription (RT) of the intermediate RNA molecules to generate ssDNA; and
(f) purifying the ssDNA with magnetic beads.

2. The method of claim 1, wherein step (b) comprises multiple primer pairs with different sequences.

3. The method of claim 2, wherein step (b) comprises a single annealing temperature to amplify DNA oligonucleotides with different sequences.

4. The method of claim 3, wherein the annealing temperature in step (b) ranges from about 60° C. to about 70° C.

5. The method of claim 4, wherein the annealing temperature in step (b) is 66° C.

6. The method of claim 1, wherein the magnetic beads in steps (c) and (f) are the same.

7. The method of claim 1, wherein step (c) comprises purifying about 1 μg to about 10 μg of DNA oligonucleotides.

8. The method of claim 1, wherein the magnetic beads in step (c) can provide DNA size selection.

9. The method of claim 8, wherein the DNA size selection yields DNA molecules ranging from about 50 bp to about 150 bp.

10. The method of claim 1, wherein step (f) comprises purifying about 100 μg to about 10 mg of ssDNA.

11. The methods of claim 1, wherein the magnetic beads in step (f) can provide DNA size selection.

12. The method of claim 11, wherein the DNA size selection yields DNA molecules ranging from about 50 bp to about 150 bp.

13. The method of claim 1, further comprising purifying the RNA products using magnetic beads after step (d) and before step (e).

14. The method of claim 1, further comprising removing remaining intermediate RNA molecules after step (e) and before step (f).

15. The method of claim 14, wherein the remaining intermediate RNA molecules are removed by alkaline hydrolysis.

16. The method of claim 1, wherein the ssDNA range from about 35 bp to about 1000 bp in length.

17. The method of claim 1, wherein the magnetic beads in step (c) comprise carboxylate groups.

18. The method of claim 1, wherein the magnetic beads in step (f) comprise carboxylate groups.

19. The method of claim 1, wherein the starting DNA oligonucleotides in step (a) is about 1-10 ng and the purification in step (d) yields about 1,000-10,000 μg IVT product.

20. The method of claim 1, wherein the ssDNA is used in an in situ fluorescence hybridization assay comprising:
contacting the sample with one or more targeting-probes, wherein each targeting-probe binds to an analyte in the sample, if present;
contacting the sample with a plurality of fiducial markers, wherein each fiducial marker comprises a plurality of ssDNA;
contacting the sample with one or more readout-probes, wherein each readout-probe independently comprises a fluorescent moiety, and wherein each readout probe binds with the one or more targeting probes, if present, and the plurality of DNA probes, thereby exhibiting one or more fluorescent signals;
imaging the one or more fluorescence signals produced by each readout-probe; and
registering the image.
